(19) United States
(12) Patent Application Publication
(10) Pub. No.: US 2005/0266044 A1
(43) Pub. Date: Dec. 1, 2005

(54) Method for modulating the surface characteristics of a device

(75) Inventor: Michael Szardenings, Wolfenbuettel (DE)

Correspondence Address: HENRY M FEIEREISEN, LLC, 350 FIFTH AVENUE, SUITE 4714, NEW YORK, NY, 10118, US

(73) Assignee: BRACHT BETEILIGUNGSGESELLSCHAFT MBH, Dusseldorf (DE)

(21) Appl. No.: 11/113,314

(22) Filed: Apr. 22, 2005

Related U.S. Application Data
(63) Continuation of application No. PCT/EP03/11560, filed on Oct. 17, 2003.

(30) Foreign Application Priority Data
Oct. 23, 2002 (EP) 02023588.3

(51) Int. Cl. A61M 36/14; A61K 51/00

(52) U.S. Cl. 424/423

(57) ABSTRACT

The invention provides a method for modulating the surface characteristics of a device, which is at least temporarily in contact with, a body fluid and/or a body tissue, which is characterized in that the device is coated at least with a ligand that binds at least one of the body's own molecules, wherein the modulation effect is caused substantially by the body's own molecule. The invention thus relies on an auto-protection principle wherein the patient's own substances are used in order to modulate the surface characteristics. Furthermore devices are disclosed which are capable of presenting a modulated surface.

### CROSS-REFERENCES TO RELATED APPLICATIONS

This application is a continuation of prior filed copending PCT International application no. PCT/EP2003/011560, filed Oct. 17, 2003, which designated the United States and on which priority is claimed under 35 U.S.C. §120 and which claims the priority of European Patent Application, Serial No. 02023588.3, filed Oct. 23, 2002, pursuant to 35 U.S.C. 119(a)-(d).

### BACKGROUND OF THE INVENTION

Medical devices that are at least temporarily in contact with a body fluid and/or body tissue as it is the case for implantation devices are commonly used by physicians. Often it is desired to allow body tissue to grow around the device's surface. For example stents are desired to be rapidly endothelized in the arterial wall, or prosthetic devices such as artificial hips are desired to be integrated into the bone structure. On the other hand certain surfaces—sometimes even of the same device—are constantly exposed to body fluids such as blood serum or body tissues where modulation is desired to avoid surgrowth or cell adhesion as far as possible.

Specifically the implantation of stents into a blood vessel illustrates the two contradictory technical needs outlined above. The outer surface of a stent is advantageously designed to be integrated by the attachment of endothelian cells into the arterial wall, whereas it is not desired that proteins or cells adhere to the stents surface on the inside of the stent which is in contact with the blood since this might lead to restenosis in the stent. In-stent restenosis narrows the diameter of the stent and is caused by An accumulation of substances that might eventually block the flow of blood through the stent. The exact reason or mechanism for the development of restenosis is unknown. What is clear though is that there is an over production of cells similar to scar tissue at the site of stent placement. Restenosis is a significant problem since additional interventional procedures or even heart surgery may be required to eliminate a re-occurring blockage. Repeated procedures typically carry a higher risk of complication.

Therefore, several approaches are known in the state of the art in order to promote the endothelialization of a stent and on the other hand to prevent restenosis.

U.S. Pat. No. 6,140,127 describes a stent which is coated with an endothelial cell specific adhesion peptide. The adhesion peptide coating promotes attachment of endothelial cells to the surface of the prosthetic object thereby resulting in rapid endothelialization of the surface of the object. It is believed that the endothelial cell specific adhesion peptide sequence increases the rate of confluence of endothelial cells along the surface of the object through migration of the endothelial cells from the environment including endothelial cells to the surface of the object. Thus the integration of the stent into the aterial wall is promoted.

U.S. 2002/0012942 A1 also describes a polypeptide coating which promotes cell adhesion to a stent surface. The described polypeptides attract cells to the surface in vivo or even promote the growth of a desired cell type on a particular surface prior to grafting. It is also mentioned that it may be advantageous to couple the peptide to biological molecules such as albumin, glycosaminoglycan or a proteoglycan. The coupling is perceived as advantageous because previous studies have shown that the coupling of synthetic polypeptides to larger carrier proteins resulted in enhanced cell adhesion activities.

U.S. Pat. No. 6,391,052 and EP 0 754 017 describe metal stents which comprise a covering sleeve of a collagen material and methods for depositing collagen coatings on a metal surface. It is further disclosed that a drug or another agent such as heparin or the like may be included in the collagen as a surface treatment which can be released after stent deployment in order to prevent e.g. thrombus formation. The described collagen coating is not limited to collagen alone but also extends to other natural materials that normally form membranes with collagen such as laminin, keratin, proteoglycans, carbohydrates, fibrin, fibronectin and the like or other materials that can be made into a film with collagen such as albumin, globulin and other blood borne proteins. Such tubular films made from any of those combinations will provide substantially the same purpose as that of pure collagen. A collagen sleeve made of such a biological material is protective and promotes cellular regrowth of endothelium.

Besides these attempts to promote integration there have been attempts in the state of the art to interfere with the overproduction of cells and to thereby reduce restenosis caused in stents or other implantation devices. One technical approach is based on placing drugs on a stent in order to repress unwanted surgrowth. These stents are called drug eluting stents. The intention is that a suitable drug is released over a certain time period to slow down the growth of unwanted cells (restenosis) and allow the vessel to heal. The problem with drug eluting stents lies in preventing the agent or agents intended for administration from being delivered or transported to undesired areas where they may have negative or damaging effects. Other difficulties arise in ensuring the permanence and the gradual release over time of active substances that can be washed away by the blood passing through the stent. Furthermore there is little or no flexibility in terms of the timing of the dissociation of the drug agent.

A further development of the idea of drug eluting stents is disclosed in U.S. Pat. No. 6,251,142. Described is an implantation device which is coated with a receptor capable of binding selectively with a ligand. The ligand is formed by combining an active principle to deliver medical effects with a substance which is capable of binding specifically to the receptor. The active principle which can have for example a therapeutic or diagnostic effect or which can improve the biocompatability of the stent is combined—without compromising its functionality and referring to known chemical reactions—with a substance capable of binding to the receptor to form the ligand. The ligand is then incorporated into a suitable preparation and is delivered to the patient into which the stent coated with the receptor has been implanted. Following administration, bonds form between the receptor and the ligand which cause the active principle to attach to the surface of the stent which is then able to function. Thus the described method allows a flexible timing of the administration of the preparations and the variety of these latter which can be matched to a stent coated with a given receptor.

WO 01/70295 describes a drug releasing stent that is coated with poly-LD-lactic acid and an endothelin-1-receptor An antagonist is attached to the surface of the stent carrying the coating. The invention involves the adhesion of BQ-123, a selective ET_{A} antagonist, on the surface of a coated coronary stent, to deliver the drugs selectively and precisely at the side of the atherosclerotic plaque, where it will be released locally over a period of time in order to limit thrombogenicity. The substances can be released in a slow controllable manner but the endothelin receptor A antagonist BQ-123 can only be delivered over a period of one month.

The methods described in the state of the art in order to modulate the surface characteristics of a device such as e.g. a stent referring to the use of immobilized or slowly released proteins or drugs suffer from many limitations. The stability of the proteins, peptides or other covering substances is often limited, compared to the expected lifetime of the clinical device. Modified, denatured or degraded covering molecules such as proteins or peptides may have adverse effects on the function of the clinical device. Furthermore the covering substances used in the state of the art are not identical to the patients' own proteins and are either of a recombinant, animal or human origin. Those solutions therefore have a high potential of either causing an immune response or being contaminated with other biological agents (for example viruses). Furthermore a therapeutic agent at one point of time will be exhausted or used up and can no longer serve it's purpose.

### SUMMARY OF THE INVENTION

Thus it is an object of the present invention to provide a device being at least temporarily in contact with a body fluid and/or body tissue which has improved characteristics.

According to a first embodiment a method for modulating the surface characteristics of a device, which is at least temporarily in contact with a body fluid and/or a body tissue is provided, which is characterized in that the device is at least partially coated at least with a ligand that binds at least one of the body's own molecules that causes a modulation effect.

The general idea of the method of the present invention is to use ligands as for example peptides, proteins or other chemical substances in order to cover the surface of a device which is in contact with the body fluid and/or body tissue and that are able to bind specific molecules as for example proteins from the body fluid and/or body tissue being in contact with the device. As a result the surface of the device will in situ be coated with certain of the body's own molecules due to the formation of a ligand/molecule binding complex. The idea of the present invention can be described as an automodulation principle wherein the patient's own molecules are referred to in order to modulate the surface characteristics of the device. The ligand can be chosen such that it binds to at least one type of body molecule carrying the desired modulation function in order to provide the desired modulation effect. Thus many different modulation effects and thus surface characteristics may be achieved.

As a further advantage of the modulating method according to the present invention the coated device (such as a stent) can obtain a considerably longer lifetime compared to the state of the art since the ligand coating itself is not in direct contact with the body fluid and/or body tissue of the patient due to the fact that the ligand binds to a body's own molecule and is thereby shielded by the bound molecule. The bound molecule can for example prevent a degradation of the ligand by enzymes from the body fluid and/or body tissue since the bound molecule is exposed to the degradation enzymes and, if at all, will be degraded first. Thus the inventive modulation method does not only allow to change the surface characteristics but at the same time to protect the underlying surface being the ligand or the device itself that may be sensitive to degradation as for example a device comprising of biological material. Degradation will successfully be prevented and the lifetime of the device itself increased. It is understood that the modulation effect may even be limited to protection of the device.

A further advantage of the method of the present invention is that the exposed surface is covered with a molecule that is unlikely to cause an immune reaction since it is derived from the body the device is in contact with. Even when using human proteins of foreign origin in the state of the art there can be a danger of immune reactions as may be with human serum albumin in case of genetic variants. Thus the problem of immunogenecity can be overcome by the present invention. Therefore the method of the present invention can also be used to disguise a foreign device or other implant which is at least temporarily in contact with a body fluid and/or body tissue. Due to the cover made up of the body's own molecules the foreign devices or other implant will be recognised by the immune system as “self” and will not be attacked.

A further advantage lies in self-repair capabilities of the system. Any molecule bound by the ligand eluting from the device's surface due to the natural half-lifetime of the ligand/molecule complex or its degradation, can immediately be replaced by an identical type of molecule from the surrounding body fluid and/or body tissue thereby maintaining the cover intact. Furthermore the stability of the chosen modulating molecule bound by the ligand can without disadvantage even be very short compared with the desired lifetime of the device since the molecule will easily be replaced. Thus there is no restriction in choice of the modulating molecule.

The device can be completely or only partially coated with the ligand, advantageously in areas where the respective modulation effect is desired. The ligand can be capable of binding only one type or several types of body molecules. It is preferred that the ligand binds its partner(s) which depict the desired surface characteristics selectively and with a high affinity.

The device may also carry more than one ligand. According to this embodiment it is possible to accomplish different surface characteristics especially in different surface areas. One area can thus be covered with a ligand which binds a body molecule depicting one characteristic and another area is covered with a different ligand binding a different body molecule with a different characteristic. This embodiment is described in detail below for the example of a stent. Further a combined coverage of surface areas is possible, for example with ligands positioned adjacent to each other. This embodiment is advantageous if the different ligands bind different body molecules thereby achieving a heterogeneous coating by the different ligand/molecule binding complexes which provide the desired surface characteristics. The different ligands can bind different types of molecules which can depict the same or a different modulation effect The embodiment with a heterogeneous coating is especially advantageous if the desired surface characteristics cannot be provided by one type of body molecule alone. A heterogeneous coating can also be achieved with one ligand binding to more than one type of body molecule. Thus according to the method of the present invention a device can be provided with numerous different surface characteristics.

A device according to the present invention can be a gadget of any kind and can be of any origin such as e.g. biological or non-biological origin and can be made of for example biological material or organic or inorganic materials. The device can specially be made of metals or metal alloys or of synthetic materials. The device can be an implantation device which is to remain in the body and which is in contact with a body fluid and/or a body tissue. The device can also be only temporarily in contact with the body as it is for example the case with chirurgical devices to hold body parts together but not be integrated in the tissue as it is the case with for example sutures, pins, plates or the like. The device can also be used extra-corporal. The device can be suitable for use in a human or an animal body. The devices can also be coated by the method according to the present invention. It is specially advantageous to cover a stent according to the method of the present invention in order to prevent in-stent restenosis.

The term ligand is to be understood in a broad sense. The ligand can for example be chosen from the group of chemical entities, peptides, polypeptides, nucleic acids and the like. The term includes naturally occurring and non-naturally occurring forms as for example D-enantiomers in case of peptides or proteins. The ligand can be attached to the surface of the device by any means known in the state of the art. It is also possible that the ligand is not attached directly to the devices surface but to a coating of the device as for example a collagen coating.

The right ligand/molecule pair can be for example found by first choosing a body molecule which depicts the desired modulation characteristics. If no appropriate ligand is known in the state of the art the body molecule can be used in order to screen chemical and/or biological libraries as for example phage and/or phagemid peptide libraries for appropriate ligands. It is preferred to chose a ligand which is highly selective for its binding partner in order to avoid the binding of “wrong” body molecules leading to undesired side-effects.

Many modulation effects may be achieved by the method of the present invention.

For example if a ligand is chosen such that a body molecule is bound, which prevents the adhesion of molecules or cells, the exposed surface will have a highly reduced or even no affinity for other proteins or cells thereby preventing their adhesion. Cell adhesion as it can lead to restenosis in stents can thus be prevented according to this embodiment. The surface of the device as e.g. of a stent can be coated with a ligand that selectively binds a blood plasma component, preferably a major component such as HSA (human serum albumin) as an example for a body molecule which prevents cell adhesion. The ligand can be a HSA specific peptide or another known compound which binds albumin from the serum. One example for a compound with HSA binding activity is the dye cibacron Blue which is for example available from Amersham Biosciences. Due to the binding of albumin to the ligand the device will be covered by a layer of albumin. Because of the shielding of the ligand by albumin, proteinases cannot attack the peptide ligand and degrade it so that the effect is long-lasting. Due to the covering of the surface with HSA the ligand is not only protected but also the attachment of other molecules or cells to the devices surface is prevented. Thereby in-stent restenosis can be prevented very efficiently.

According to a further embodiment of the present invention the ligand is chosen such that molecules are bound which promote cell attachment and/or growth in permanent implants. A ligand with sufficient affinity but without inherent biological activity can enhance the concentration of for example the patient's own growth hormones by binding to them in the vicinity of such devices. Thereby it is possible to for example promote the integration of a device in the surrounding tissue. In case the implantation device is a stent which is suppose to be implanted in the arterial wall, the bound body molecule may promote endothelialization.

According to another aspect of the present invention a device which is at least temporarily in contact with a body fluid and/or the body tissue is provided which is characterized in that the device is at least partially coated at least with a ligand that is capable of binding one of the body's own molecules, the molecule then causing a modulation effect. The embodiments and advantages of a device carrying such a coating are as outlined above.

Devices which are in contact with the blood of a patient and where cell adhesion is supposed to get prevented are coated preferably with a ligand that selectively binds albumin or another major protein component of the blood plasma (i.g. alpha2macroglobulin). But also other body's molecules can be used which prevent the adhesion of molecules and/or cells.

In case rapid integration of the device in the body of the patient is desired, the device can be coated with a ligand that binds to body molecules promoting the adhesion of molecules or cells in order to for example promote the integration of the device into a for example bone structure or the endothelialization of a stent into an arterial wall.

In order to combine both effects and in order to provide a stent which overcomes the problems known in the state of the art, it is therefore advantageous to use a stent which is coated on the inside with a ligand that binds to a body's molecule of the patient that prevents cell adhesion. For example a peptide or compound can be used which binds to HSA. The stent can further be coated on the outside with a ligand that binds to a body's molecule promoting the adhesion of molecules. Thereby the attachment of cells and thus the endothelialization of the stent into the arterial wall is enhanced. This would be an example for a device carrying more than one ligand and with more than one surface characteristics in defined areas.

The described embodiments can be used in order to provide a method for lowering the restenosis rate in stents which is characterized in that a stent is used which is at least partially covered with a ligand that binds to a body's own molecule that prevents adhesion of molecules such as cells. A stent as described above can be used in combination with the method.

The devices according to the present invention can also include features already known in the state of the art. For example it is possible to additionally attach or incorporate a therapeutic or diagnostic agent to the device's surface in order to additionally apply a desired drug at the place of implantation. For the use with for example stents a combination is possible where the inside of the stent is coated according to the present invention in order to prevent cell adhesion and restenosis and the outside is treated according to a method described in the state of the art as described above in order to promote endothelialization since the process of endothelialization takes place only over a limited time period. Afterwards the stent is integrated in the aterial wall and the promotion of endotheliazation is not longer necessary.

### BRIEF DESCRIPTION OF THE DRAWING

The invention will be described in more detail for a stent as an example of the inventive device.

FIGS. 1a and 1b show the state of the art;

FIGS. 2a and 2b outline the principle of the present invention.

### DETAILED DESCRIPTION OF PREFERRED EMBODIMENTS

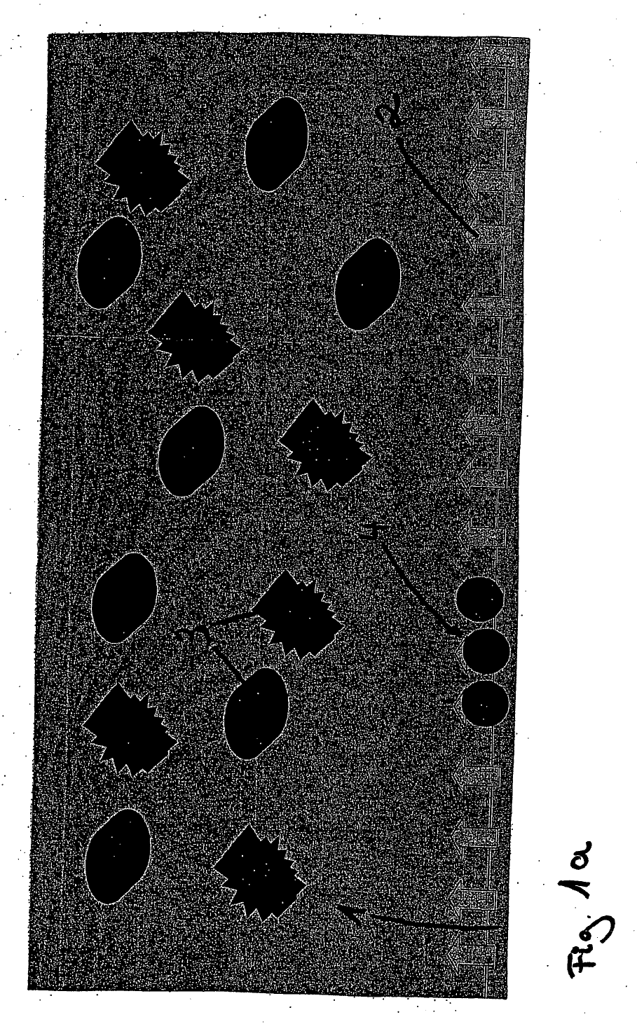

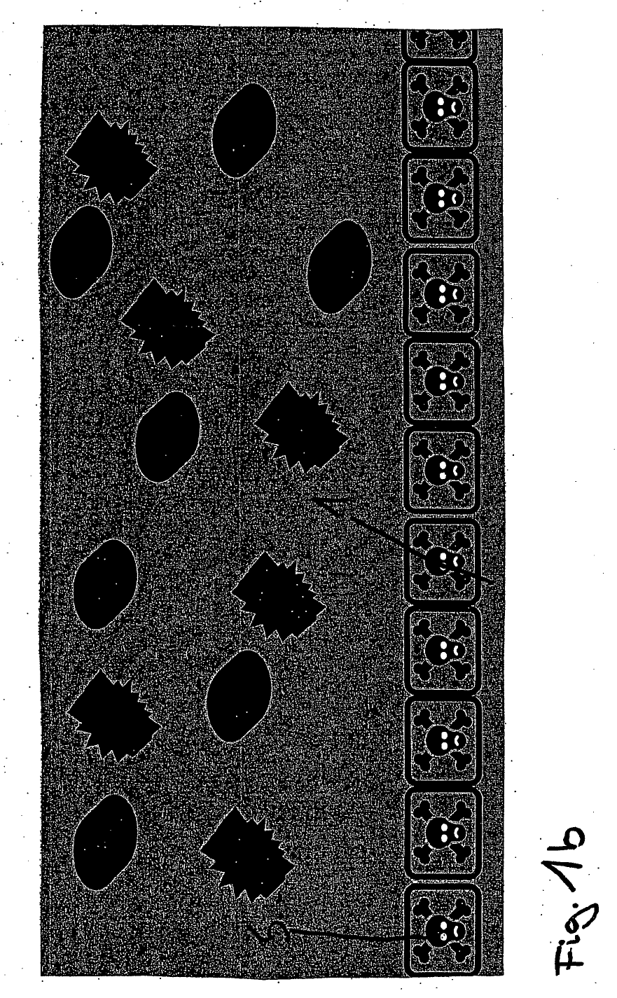

FIG. 1 describes the method known in the state of the art, wherein the surface of the stent 1 is coated with non-adhesive agents 2 in order to prevent cell attachment to the surface. The example describes the implantation of a stent 1 in a blood vessel. Shown is the inside of the stent which is in contact to the blood serum wherein 3 indicates serum molecules. After exposure of the stent surface 1 to the plasma the covering protective agents 2 can be destroyed by enzymes and/or chemicals as indicated by 4. Thus the stent surface 1 is then directly exposed to the serum and cell adhesion starts at the exposed surface areas possibly leading to in-stent restenosis.

FIG. 1b describes the protection of the stent surface 1 which is in direct contact to the blood by toxins 5. The toxins 5 can for example be attached to or be included in the coating of a stent. The toxin 5 will slowly be released over a period of time thus also leaving the surface unprotected allowing cell adhesion to take place and thus in-stent restenosis to develop.

As will be evident to a person skilled in the art from the described examples surfaces exposed to body fluid such as blood serum are permanently attacked by degrading enzymes. Even protective materials such as toxins or the like will lose their activity over time which is a disadvantage since implantation devices are meant to be long lasting. Also chemical substances such as polymers will eventually be degraded and can not protect the device's surface for a long time. Since toxic compounds will exhaust and peptides or proteins will be degraded after a while there is no real efficient long-term protection known in the state of the art. Furthermore it is not desireable to bring a patient's body in contact with chemicals such as drugs and/or toxic compounds.

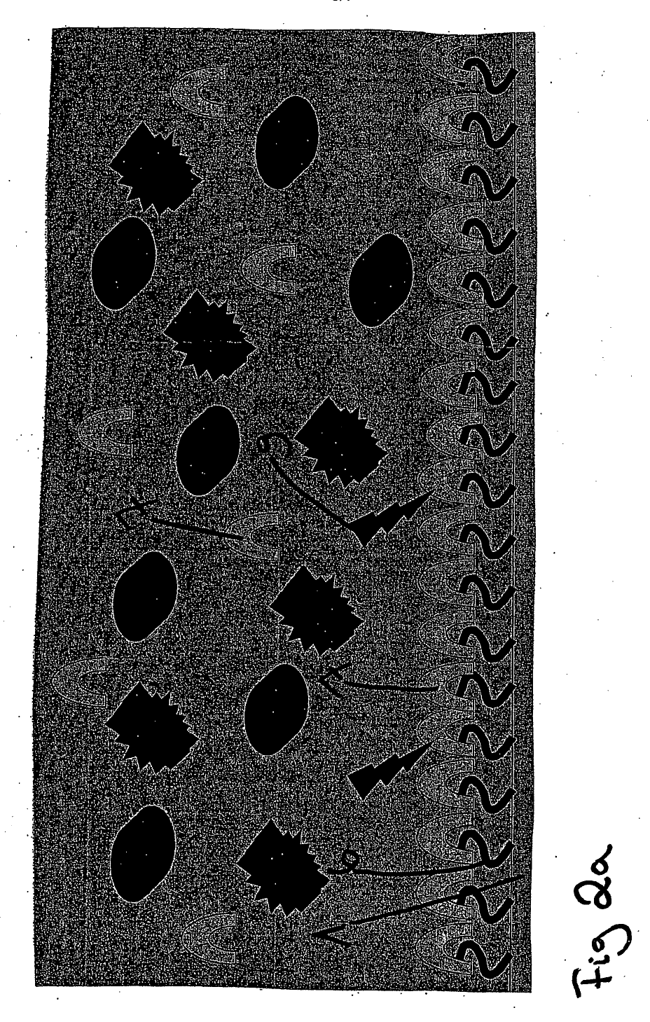

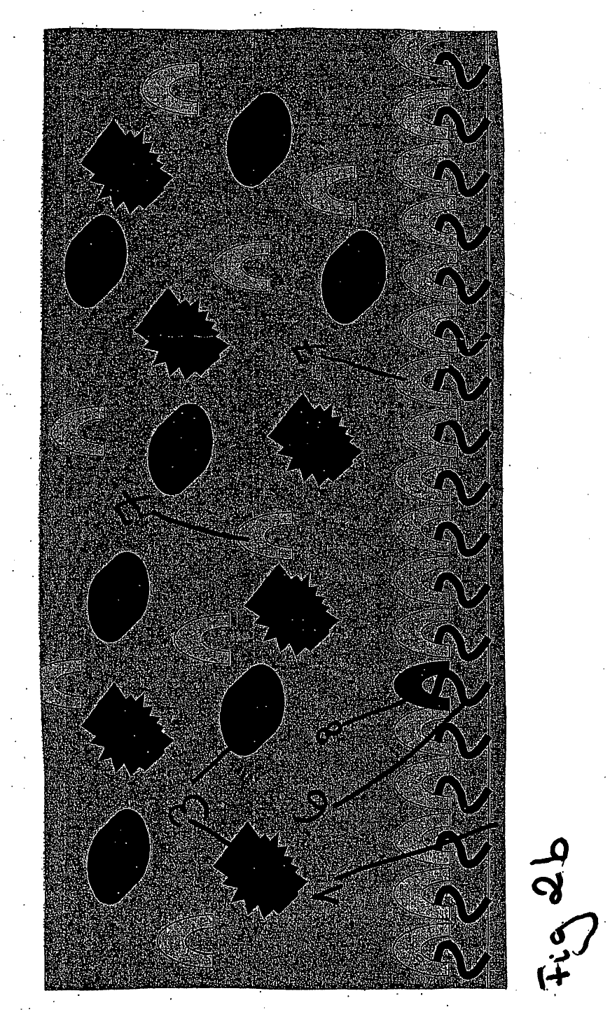

FIG. 2a shows the principle of the present invention. It is based on automodulation by using the body's own molecules in order to modulate respectively protect and/or the devices surface. In this example a protein is used for coating the plasma exposed surface of a stent 1. The surface of the stent 1 is covered with a HSA specific peptide ligand 6 which will bind albumin 7 from the serum. Thus the surface of the stent 1 is modulated by the albumin 7 coating which is produced by the binding of the albumin to the albumin specific ligand 6. A modulated surface is generated wherein the body's own molecule (albumin) causes the modulation effect. Due to the albumin coating 7 degrading enzymes such as proteinases 9 cannot attack the peptide ligand 6 which is therefore protected and will have a longer lifetime. Furthermore, provided that sufficient amounts of ligands are immobilized on the surface, for example more than one peptide-ligand molecule on the surface area is covered by a single molecule of for example albumin, even partial degradation of the peptide will not decrease complete protection of the surface. By the use of albumin 7 the adhesion of cells is effectively prevented thus preventing restenosis in a stent.

FIG. 2b resembles FIG. 2a and illustrates the self-repairing nature of the present invention. As soon as a bound albumin 8 dissociates or is denatured by degradive enzymes the peptide ligand 6 is released and can bind a new HSA molecule 7 from the serum. The use of HSA is especially advantageous due to the high serum concentration of albumin which allows the rapid formation of ligand/albumin binding complexes. Thus an effective and rapidly regenerateable protection is achieved and the modulated surface will always be replaced and repaired increasing lifetime of the stent and efficient restenosis protection.

While the invention has been illustrated and described in connection with currently preferred embodiments shown and described in detail, it is not intended to be limited to the details shown since various modifications and structural changes may be made without departing in any way from the spirit of the present invention. The embodiments were chosen and described in order to best explain the principles of the invention and practical application to thereby enable a person skilled in the art to best utilize the invention and various embodiments with various modifications as are suited to the particular use contemplated.

What is claimed as new and desired to be protected by Letters Patent is set forth in the appended claims and includes equivalents of the elements recited therein:

1. A method for modulating the surface characteristics of a device, which is at least temporarily in contact with a body fluid and/or a body tissue, characterized in that the device is at least partially coated at least with a ligand that binds one of the body's own molecules, wherein the molecule has a modulation effect.

2. The method according to claim 1, wherein the device is coated with more than one ligand wherein the different ligands are concentrated in different surface areas.

3. The method according to claim 1, wherein a body molecule with certain modulation characteristics is chosen in a first step and in a second step an appropriate ligand is chosen which selectively binds the chosen body molecule.

4. The method according to claim 1, wherein the ligand is chosen from the group of chemical entities, peptides, polypeptides or polynucleotides.

5. The method according to claim 1, wherein the device is coated with a ligand that binds a blood plasma component, preferably human serum albumin (HSA).

6. The method according to claim 1, wherein the device is coated with ligands that bind molecules that decrease and/or prevent the attachment of further molecules.

7. A device which is at least temporarily in contact with a body fluid and/or a body tissue, characterized in that the device is at least partially coated at least with a ligand that is capable of binding at least one of the body's molecules causing a modulation effect.

8. A device according to claim 7, wherein the device is a stent.

9. A device according to claim 7, wherein the device is coated with a ligand selected from the group of chemical entities, peptides, polypeptides and/or polynucleotides.

10. A device according to claim 7, wherein the device is coated with a ligand that selectively binds a blood plasma component, preferably HSA.

11. A device according to claim 7, wherein the device is coated on the inside with a ligand that binds to a body molecule which prevents the adhesion of molecules.

12. A device according to claim 7, wherein the device is coated on the outside with a ligand that binds to a body' molecule promoting the integration of the device in the surrounding tissue.

13. A device according to claim 7, wherein the device comprises a surface coating and wherein the ligand is attached to the surface coating.

14. A device according to claim 7, characterized in that the device comprises active agents.

15. A device according to claim 7, characterized in that device comprises more than one ligand, wherein the different ligands are concentrated in different surface areas of the device.

16. The use of a stent which is at least partially covered by a ligand that binds to a body molecule that prevents the adhesion of molecules and/or cells to the surface of the stent for preventing or reducing in-stent restenosis.

17. The use according to claim 16, characterized in that a stent coated with a ligand selected from the group of chemical entities, peptides, polypeptides and/or polynucleotides.

18. The use according to claim 16, wherein said body molecule is albumin.
